(19) United States
(12) Patent Application Publication
(10) Pub. No.: US 2015/0110670 A1
(43) Pub. Date: Apr. 23, 2015

(54) DECONTAMINATION OF ISOLATION ENCLOSURES

(71) Applicant: NOXILIZER, INC., Baltimore, MD (US)

(72) Inventors: David Opie, Baltimore, MD (US); Evan M. Goulet, Baltimore, MD (US); Blaine G. Doletski, Catonsville, MD (US); William E. Waters, Millersville, MD (US)

(21) Appl. No.: 14/239,595

(22) PCT Filed: Aug. 17, 2012
(86) PCT No.: PCT/US2012/051425

§ 371 Date: Jan. 5, 2015

Related U.S. Application Data
(60) Provisional application No. 61/525,424, filed on Aug. 19, 2011.

(51) Int. Cl. A61L 2/20

(52) U.S. Cl. CPC A61L 2/20
USPC 422/28

(57) ABSTRACT

A system and method for decontamination of isolation enclosures includes a recirculating isolator configured to allow injection of a sterilant gas into the isolator. Levels of humidity and sterilant gas are selected to avoid condensation of either within the isolator. In an embodiment, a positive pressure is maintained throughout the sterilization process.

This application claims priority to U.S. Provisional Patent Application 61/525,424 filed Aug. 19, 2011, which is incorporated by reference in its entirety herein.

### BACKGROUND OF THE INVENTION

1. Field

This application relates generally to sterilization systems and more particularly to sterilization systems for use in decontamination of isolators.

2. Description of Related Art

Isolators are structures designed to maintain a sterile environment for manufacturing or laboratory activities where contamination risk must be mitigated. As an example, isolators are used in the pharmaceutical industry to provide sterile environments for drug processing and/or sterility assurance testing with minimal risk of contamination by viable microorganisms. They are typically operated at a slight positive pressure to prevent introduction of outside contaminants via leakage pathways into the enclosure. As a result, isolators are not amenable to use of vacuum cycles during decontamination operations.

In an open loop sterilization or decontamination system, sterilant is added to a chamber and then withdrawn from the chamber after a dwell period. A sterilizer unit that employs a vacuum phase, as is used for medical equipment, is an example of an open loop system. A closed loop system is one in which gas from the enclosure is recirculated for the purpose of adding or removing sterilant or humidity. Typically, a closed loop system is used when the enclosure cannot support the forces associated with creating a vacuum within the enclosure. Certain gas delivery systems, as would be used with an isolator, are an example of a closed loop system. In U.S. Pat. No. 4,909,999 Cummings, et al., describe a method of introducing a sterilant vapor to a room or chamber in which the sterilant vapor is formed from hydrogen peroxide and water, and using a recirculating gas circuit. The removal of the sterilant involves the use of heat to rapidly decompose the hydrogen peroxide.

For sterilization and decontamination of isolator enclosures, vapor hydrogen peroxide (VHP) is most widely used as the sterilant. Generally, there are two types of hydrogen peroxide-based systems described: systems that dehumidify the enclosure gas with dryers, and systems that humidify the enclosure gases in order to controllably form some water and sterilant vapor condensation. For an example of using a dehumidification phase, U.S. patent application Ser. No. 11/421,265 teaches use of a dryer in a dehumidification phase. After dehumidification, conditioning is performed and VHP is injected at a high flow rate. Systems that use a dryer for dehumidification are also described in U.S. Pat. No. 5,173,258 and U.S. Pat. No. 5,906,794.

It has been shown that excess moisture buildup in an enclosure can hinder the sterilization process and rapid removal of the sterilant. As hydrogen peroxide degrades into oxygen and water, water content in the enclosure tends to increase. To avoid the problem of excess water caused by using hydrogen peroxide as a sterilant, Childers et al., describe a method of drying the gas circulating in the chamber in U.S. Pat. No. 5,173,258.

Using chlorine dioxide as the sterilant generally requires high humidity, resulting in the presence of excess water. For example, chlorine dioxide decontamination and sterilization is described US Patent Application No. 2009/0246074 A1, by Nelson, et al., wherein high levels of humidity are required. Such high levels of humidity tend to require extended aeration periods.

### BRIEF SUMMARY OF THE INVENTION

A system and method for decontamination of isolation enclosures includes a recirculating isolator configured to allow injection of a sterilant gas into the isolator. Levels of humidity and sterilant gas are selected to avoid condensation of either within the isolator. In an embodiment, a positive pressure is maintained throughout the sterilization process.

### BRIEF DESCRIPTION OF THE DRAWINGS

FIG. 1 is a schematic illustration of a system in accordance with an embodiment of the invention;

FIG. 2 is a graph illustrating degrees of lethality for two exposure cycles plotting negative biological indicators versus sterilant injection time;

FIG. 3 is graph illustrating degrees of lethality for a series of exposures plotting negative biological indicators versus dose, where dose is expressed as a product of amount of sterilant and time;

FIG. 4 is a graph illustrating degrees of lethality plotting log surviving population versus sterilant injection time;

FIG. 5 is a graph illustrating FTIR measurements of water and NO₂ profiles during a sterilization cycle;

FIG. 6 is a graph illustrating NO₂ concentration versus time in a purge cycle; and

FIG. 7 is a graph illustrating a relationship between NO₂ removal mechanisms in a purge cycle.

### DETAILED DESCRIPTION OF THE INVENTION

In an embodiment in accordance with an aspect of the present invention, nitrogen dioxide (NO₂) is used as the sterilant gas. Generally, NO₂ has a low boiling point and high vapor pressure at room temperature, which the inventors have found makes it particularly well suited to sterilization or decontamination of enclosures. Use of a low boiling point sterilant may allow handling in either liquid or gaseous form, as well as avoiding a need to generate extreme temperatures or requiring the isolator to be made using highly heat or cold resistant materials. Furthermore, low boiling point sterilants will not tend to condense on surfaces of the enclosure, reducing the potentially dangerous deposition of residual sterilant.

In embodiments, sterilant may be introduced to the enclosure directly, by way of a gas injection system. Alternately, sterilant may be introduced into a recirculating gas stream.

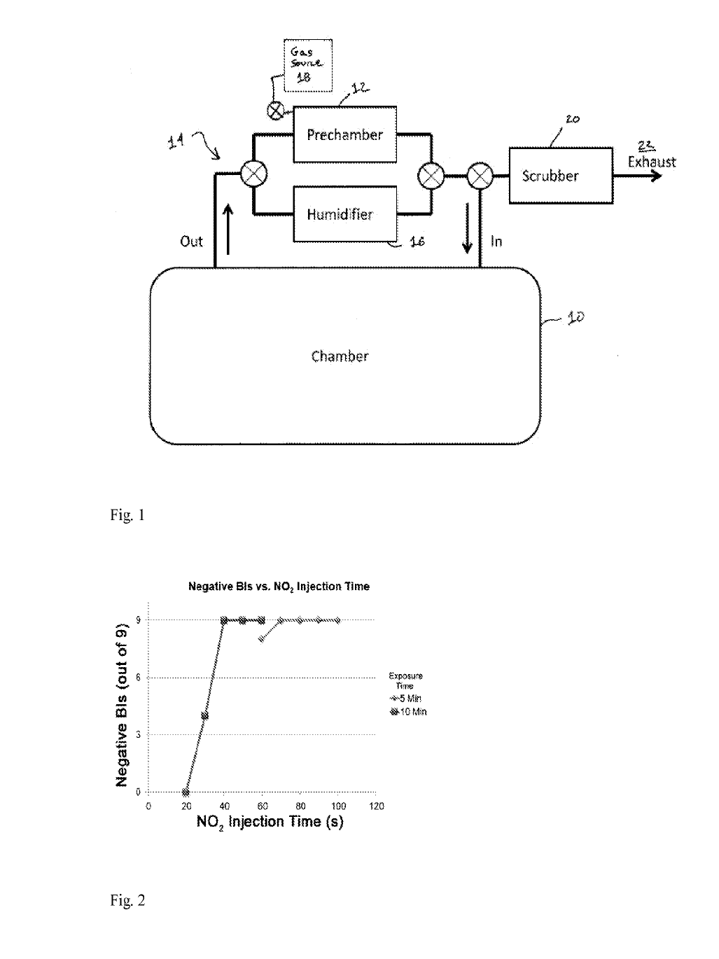

In an embodiment, illustrated in FIG. 1, sterilant is metered using a pressure and volume measurement of the sterilant gas. An isolator (or other chamber to be sterilized) 10 is in fluid communication with a pre-chamber 12. With low boiling point and high vapor pressure sterilants, the target concentration needed for effective decontamination may be much lower than the saturation vapor pressure of the gas. As a result, metering the gas by measuring pressure of the gas in a pre-chamber with a known volume gives a convenient means of dose control. A pre-chamber process of this type is described in U.S. patent application Ser. No. 12/710,053, hereby incorporated by reference in its entirety.

A recirculating gas flow circuit 14 may be used to flush the contents of the pre-chamber (or, gas generating chamber) into the enclosure. This approach does not require the addition of heat to generate the NO₂ gas, it can be generated at room temperature.

An optional humidifier 16 may be included within the recirculating gas flow circuit 14. A sterilant gas source 18 is in communication with the pre-chamber 12.

An alternate approach to introducing the sterilant gas to the chamber or enclosure is the use of one or more injection nozzles that directly introduce the sterilant into the enclosure volume or recirculating gas stream. With a low temperature boiling point sterilant gas, like nitrogen dioxide, nozzles at room temperature, or slightly elevated temperature, may be used to dose the liquid sterilant directly into the chamber. Where a temperature of the sterilant is close to or above the boiling point, sterilant would vaporize as it exits the nozzles.

In an embodiment, liquid nitrogen dioxide may be metered by weight or volume prior to introduction into the enclosure, recirculating gas stream, or gas generating pre-chamber. In another embodiment, a chemical composition that generates NO₂ may be positionable within the pre-chamber where it may be activated to generate the NO₂ for sterilization. The gas delivery may be accomplished by using a DOT approved cylinder holding a quantity of liquid NO₂ (which is actually the dimer N₂O₄).

In an embodiment, nitric oxide (NO) can be added to the recirculating gas stream or gas generating prechamber. NO can be stored as a compressed gas in gas cylinders. The gas will mix with air in the prechamber, in the reciculating gas stream, and/or in the enclosure. Upon mixing with air, the NO will react with oxygen to form NO₂.

In an embodiment, concentrations of sterilant and temperatures are selected such that the sterilant does not condense. Sterilant condensation can tend to increase the time needed to aerate the chamber of residual sterilant gas, as the condensed sterilant does not rapidly evaporate. Certain corrosive sterilants (such as hydrogen peroxide) may be damaging to materials within the isolator, or can cause injury to personnel who come into contact with condensed sterilant.

Likewise, condensing levels of humidity tend to lead to sterilant condensation. If liquid water forms on surfaces, the sterilant will tend to form a mixture (solubilize) with the water, increasing the amount of condensed sterilant. This will tend to further increase the time needed to aerate the enclosure. Therefore, embodiments employ humidity levels less than a condensing level. In an embodiment, humidity within the isolator is controlled to between 30 and 90% relative humidity, and particularly, between 70 and 85% relative humidity. In a particular embodiment, the isolator is controlled to between 55 and 70% relative humidity.

Experiments were performed to simulate effectiveness of methods as described herein. A test chamber was operated in a manner that simulated an industrial isolator system, by employing cycles with minimal changes in pressure during gas introductions. In one testing protocol, sterilant concentrations necessary to achieve a six-log reduction in spore population on commercial biological indicators (BIs) at exposure times of 5 and 10 minutes were determined.

In another testing protocol, the ability of a dry air purge to clear sterilant from the chamber in a timely manner was demonstrated. A purge of the chamber (enclosure) introduces air that does not contain sterilant into the chamber as gas containing sterilant is removed from the chamber. No vacuum was applied either prior to sterilant gas introduction or for sterilant gas removal. Instead, the cycles described herein rely on an exhaust port (or vent valve), which was left open, permitting gas to escape from the chamber, thereby maintaining a constant chamber pressure. In this manner, as gas is added to the chamber, gas displaced by the added is exhausted from the chamber. This approach simulated the gas addition and removal as would be observed in the case where a recirculating gas circuit would be used to add or remove sterilant and humidity from the enclosure.

The results of these tests are discussed below and demonstrated the ability to humidify the chamber and add lethal amounts of NO₂ gas with minimal pressure increases. Cycle conditions that sterilized commercial biological indicators (BIs) with 5×10⁶ CFUs were selected, using exposure times of 5 and 10 minutes. The ability to purge the chamber to less than 1 ppm NO₂ utilizing a dry air flush of approximately 30 minutes was also demonstrated.

The specific exposure cycles performed during these tests are shown in Table 1. The duration during which NO₂ was added to the chamber was varied as a means of varying the resulting concentration of NO₂ in the chamber during the exposure dwell phase of the cycle. Biological indicators were placed in the chamber during each cycle in order to determine the exposure conditions that yielded a six-log spore population reduction on commercial biological indicators (BIs) exposed.

TABLE 1
The NO2 injection times are given for each of the ten cycles
at 5-minute and 10-minute exposures. Because the experimental
open to the atmosphere via vent valves, time, rather than pressure,
was used the NO₂ gas additions.

| | 5-Min. Exposure | | 10-Min. Exposure | |
|---|---|---|---|---|
| | Cycle No. | NO₂ Injection (sec) | Cycle No. | NO₂ Injection (sec) |
| NO₂ | 1 | 60 sec. | 6 | 20 sec. |
| Injection | 2 | 70 sec. | 7 | 30 sec. |
| Time | 3 | 80 sec. | 8 | 40 sec. |
| | 4 | 90 sec. | 9 | 50 sec. |
| | 5 | 100 sec. | 10 | 60 sec. |

Prior to starting each cycle, 13 BIs were placed in the chamber. The BIs were widely distributed on the chamber shelf. Nine of these 13 BIs were used for fraction negative tests, where, after exposure, each BI was placed in test tubes containing tryptic soy broth and incubated. The incubated test tubes that exhibited turbidity after an appropriate incubation time were judged to be ‘positive’, and to have had viable spores on the BI placed in that test tube. Test tubes that did not exhibit growth were deemed to be ‘negative’ for surviving (viable) spores on the BIs in that test tube. The number of negative and positive BIs for each cycle were recorded.

The results of the fraction negative testing are shown by the number of negative BIs in Table 2. With the 5-min exposures, one cycle (Cycle No. 1) had one positive BI and all other 5-min cycles were negative. For the 10-min exposures, Cycles 6 and 7 resulted in nine and five positive BIs, respectively. The other three cycles yielded complete sterilization of the nine BIs. In addition to the nine BIs used for fraction negative testing, four BIs were included in each cycle for direct enumeration of surviving CFUs. The results of the plate counts are shown as the average log of recovered CFUs per BI in Table 2.

The results of the fraction negative BI testing are plotted in FIG. 2. As the NO₂ injection time was increased, thereby increasing NO₂ concentration in the chamber, lethality was increased. Each G. stearothermophilus BI had a population of approximately 5×10⁶ CFU. Therefore, a cycle with nine negative BIs achieved at least a 6.7-log reduction in spore population. The average RH achieved in the all of the cycles was 81%. At this humidity level, the 5-minute exposure required an NO₂ injection time of 70 s (Cycle 2) to sterilize all nine BIs. This corresponded to an NO₂ injection concentration of approximately 8.2 mg/L. The 10-minute exposure cycle required 40 s of NO2 injection, or approximately 4.7 mg/L NO2 (Cycle 7).

TABLE 2
The results of microbiological testing are shown below.
Nine BIs included in each cycle were tested via a fraction negative
method, and four BIs were included for direct enumeration of surviving
CFUs.

| 5-Min Exposures | | | | 10-Min Exposures | | | |
|---|---|---|---|---|---|---|---|
| | NO₂ | Fraction | Avg | | | Fraction | Avg |
| | Injec- | Negative | Log | | NO₂ | Negative | Log |
| Cycle | tion | BIs | CFUs | Cycle | Injection | BIs | CFUs |
| 1 | 60 | 89% | 0 | 6 | 20 | 0% | 4.7 |
| 2 | 70 | 100% | 0 | 7 | 30 | 44% | 4.4 |
| 3 | 80 | 100% | 0 | 8 | 40 | 100% | 0 |
| 4 | 90 | 100% | 0 | 9 | 50 | 100% | 0 |
| 5 | 100 | 100% | 0 | 10 | 60 | 100% | 0 |

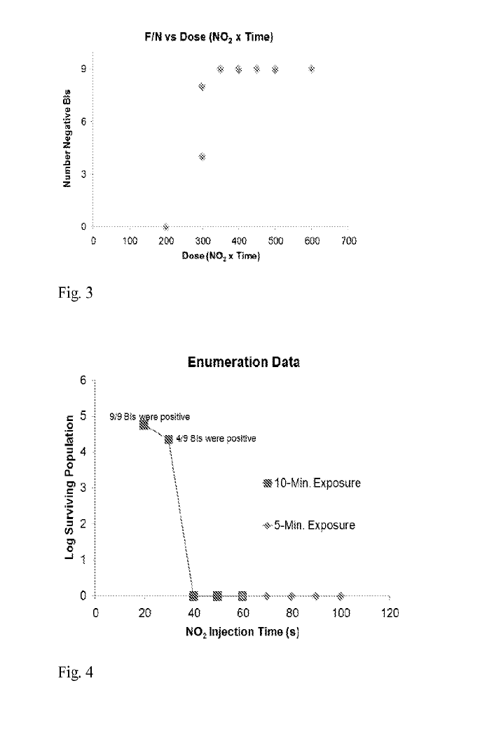

If one treats the overall NO₂ dose of a given cycle as the product of NO₂ injection time multiplied by exposure time, then the fraction negative data for all cycles (both 5 minute and 10 minute exposure times) can be plotted on one curve as the number of negative BI's versus dose, as is shown in FIG. 3. From FIG. 3, one can see that there was a dose response to the fraction negative test data. This fact may aid in predicting cycle parameters for future testing.

Four of the BIs from each cycle were used for direct enumeration of the surviving spores. These BIs were processed with a spore recovery procedure that is known to collect a large percentage of the spores from the BI carrier. The collected spores were grown on agar plates in a manner that permitted counting of the spores collected by counting the colonies that grow on the agar plates. The resulting colony forming units (CFUs) on each agar plate were counted and the average number of CFUs per BI per cycle were recorded, and plotted against the NO₂ injection time. FIG. 4 shows a plot of recovered CFUs per BI versus NO₂ injection time.

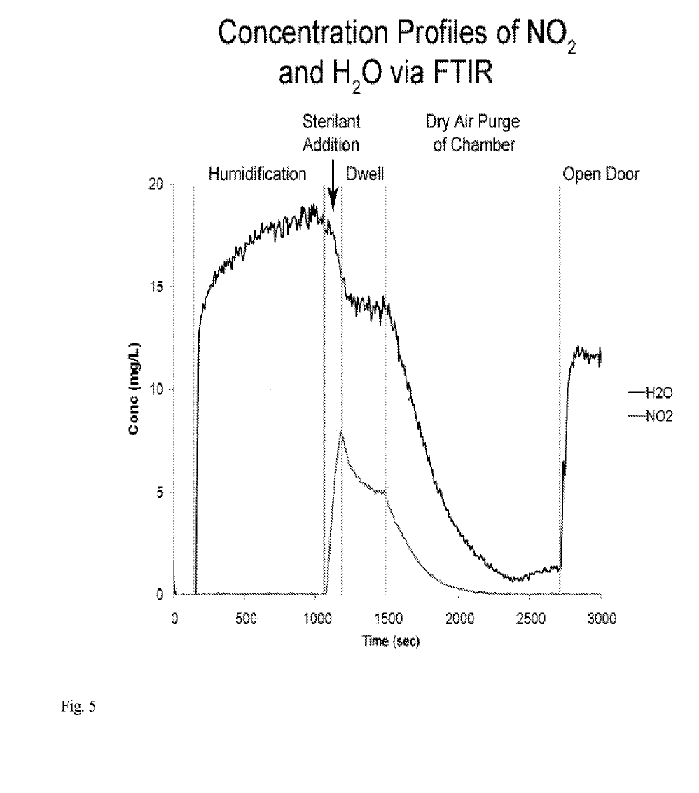

A Fourier Transform Infrared (FTIR) spectroscopy system was used to monitor both the NO₂ and H₂O gas concentrations in the chamber during each cycle. A typical concentration profile for H₂O and NO₂ during one of the cycles is shown in FIG. 5. The humidification of the chamber was carried out first, followed by the introduction of the NO₂ sterilant. After a decontamination dwell period, 5 min in the case of this particular cycle shown, a flush of dry air was performed to displace the NO₂ until safe limits were reached. The maximum H₂O and NO₂ levels, maximum RH, and the final H₂O and NO₂ levels for cycles one through seven are reported in Table 4.

The maximum NO₂ concentration for Cycle 2 was 6.6 mg/L, which was lower than the theoretical maximum of 8.2 mg/L. This apparent reduction in sterilant concentration was attributed to two factors. The first factor was the open vent valve, intended to simulate a recirculating isolator system. This would have allowed some percentage of the sterilant to be vented out the chamber during filling, as this part of the cycle was done under a slight positive pressure, as is common with industrial enclosures. The second factor that contributed to the apparent reduction in sterilant concentration was the interaction of NO₂ gas with H₂O. In FIG. 5, one can see that the NO₂ sterilant concentration continued to decrease throughout the dwell period (although the gas concentration is approaching an equilibrium concentration).

TABLE 3
The maximum and final values for both NO₂ and H₂O are
reported along with the % RH for each cycle.

| | H₂O | H₂O | RH | NO₂ | NO₂ |
|---|---|---|---|---|---|
| | Max | Final | Max | Max | Final |
| Cycle | (mg/L) | (mg/L) | (%) | (mg/L) | (mg/L) |
| 1 | 17.4 | 14.9 | 80 | 4.5 | 2.1 |
| 2 | 17.7 | 14.6 | 83 | 6.6 | 4.0 |
| 3 | 17.8 | 16.3 | 88 | 7.1 | 3.0 |
| 4 | 19.0 | 14.9 | 78 | 7.29 | 4.4 |
| 5 | 19.1 | 14.0 | 80 | 8.0 | 5.0 |
| 6 | 19.6 | 17.6 | 82 | 2.3 | 1.3 |
| 7 | 19.7 | 17.6 | 78 | 3.20 | 1.5 |

A combination of FTIR spectroscopy and electrochemical sensors (EC cells) was used to measure the NO₂ levels in the exhaust gas from the test unit chamber on a cycle that employed the exposure condition described by Cycle 4 in Table 2. At the end of the exposure time, a 60 minute purge of dry air at a rate of 40 LPM was used to clear the test unit chamber of sterilant. This purge rate was equal to approximately one chamber volume exchange per minute. The test chamber was 44 L in volume.

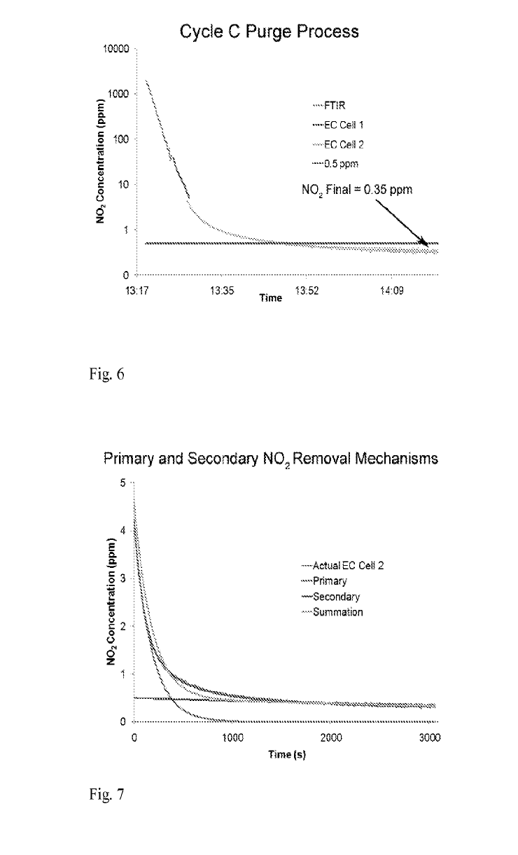

The FTIR was used to measure the exhaust gas from the test unit until the concentration of NO₂ in the gas fell below 100 ppm. At that point, the exhaust gas was directed to EC Cell 1, which had been calibrated for concentrations from 0 ppm to 100 ppm. When the NO₂ concentration of the exhaust gas dropped below 10 ppm, the gas was shifted towards EC Cell 2, calibrated for 0 ppm to 10 ppm NO₂ measurements, for the duration of the purging process. FIG. 6 shows the measured NO₂ concentration throughout the purging process.

An exponential fit of the FTIR measurements yields an NO₂ removal rate of:

y=2112e^{-0.013x}

Upon switching to EC Cell 1, the exponential fit of the NO₂ removal rate fit the following equation:

y=33.67e^{-0.0097x}

These two measurements were similar in the rate of reduction indicating that the dry air purge of gas from the test unit chamber was the primary dynamic of NO₂ removal.

If one looks at the measurement data from EC Cell 2, the first three minutes of data (minutes 8 through 11 of the purge) followed an exponential decay pattern that eventually changed slope to a point where the rate of NO₂ removal was slowed significantly. The first three minutes of EC Cell 2 data was fit to:

y=4.18e^{-0.0056x}

While the significantly slower rate of NO₂ removal could be fit to the following equation:

y=0.50e^{-0.00013x}

The change in slope of the curve may be explained by a transition from the primary NO₂ removal dynamic to a secondary dynamic. The data from EC Cell 2 were used to model the transition from the primary NO₂ removal dynamic to the secondary dynamic. A simple addition of the primary and secondary fits from EC Cell 2 was found to provide a good match to the actual EC Cell 2 data. This model is described by the following equation, which is the summation of the primary and secondary fits.

[NO₂]=4.18e^{-0.0056t}+0.50e^{-0.00013t}

There was no obvious evidence for a tertiary dynamic or other unaccounted for mechanism in the NO₂ removal process. The primary and secondary fits, the sum of the two fits, and the actual EC Cell 2 data are shown in FIG. 7. One can see that the above model fits the actual EC Cell 2 data fairly well.

The inventors propose that the most likely source of the secondary NO₂ removal dynamic is related to the structure of the chamber walls. Specifically, the Teflon coating of the test unit's chamber and the Teflon shelf within the chamber are at least partially permeable to NO₂ and will tend to absorb a fraction of the NO₂ gas introduced to the chamber. The chamber coating is approximately 3200 in², while the shelf contributes roughly 600 in². It is proposed that as the purge process progressed, the NO₂ desorbed from the surface as it diffused out of the Teflon matrix. This secondary dynamic proved to be slower than the primary dynamic of NO₂ displacement.

The final NO₂ concentration reached after 60 min of purging was approximately 0.35 ppm.

In view of the secondary mechanism described above, it may be useful to construct an isolator in accordance with an embodiment using materials selected to have low permeability to NO₂. Such low permeability materials include glass and stainless steel. Furthermore, smooth surfaces may be used to discourage adherence or embedding of contaminant, as well as reducing adsorption of NO₂ or water. The relatively small surface area of more permeable polymers is not expected to influence this rapid aeration rate.

In embodiments as described above, gas ports are described for injection of sterilant gas, air, and/or humidity. In this regard there may be multiple gas ports or all gases may be introduced through a common port. Likewise the gases may pass through a manifold to improve distribution within the chamber. In this approach, it may be useful to include a valving system such that individual lines are separately controllable.

Embodiments may include temperature controls including, for example, temperature sensors, heaters and/or coolers. A humidity sensor may also be included to allow a feedback control of system humidity conditions. In an embodiment, the source of humidity is controlled to provide humidity in vapor form and to avoid delivery of water particles, which may tend to interfere with aspects of the sterilization process.

As will be appreciated, the system described may find application with a variety of gaseous sterilants, though the inventors have found particular advantage in use of nitrogen dioxide gas. In use, a sterilization cycle with NO₂ employs between about 5 mg/L to 20 mg/L (roughly 0.25% to 1% at ambient pressure).

A scrubber system 20 may be located in the gas recirculation circuit, and used to capture the NO₂. Alternately, it may be located in an exhaust pathway 22 used in the purge cycle as shown in FIG. 1. In an embodiment, the scrubber system may be configured to reduce the NO₂ concentration in the pump exhaust to <1 ppm. By way of example, exhaust gases may be passed through a permanganate medium to capture the NO₂. Permanganate is a good adsorber of NO₂, and once saturated, is landfill safe. The pumping rate for evacuation pumps may be selected to be sufficient to evacuate the chambers within one minute, or more particularly, within 30 seconds.

A user interface, not shown, may be incorporated allowing for programming of aspects of the system. This may include, for example, timing of stages (i.e., conveyor speed), dosage of sterilant, humidity and/or temperature, and others. The user interface may also include displays for providing a user with information regarding the defined parameters and/or indications of operating conditions of the system. Controllers can be based on computers, microprocessors, programmable logic controllers (PLC), or the like.

Although the invention has been described in detail for the purpose of illustration based on what are currently considered to be the most practical and preferred embodiments, it is to be understood that such detail is solely for that purpose and that the inventions are not limited to the disclosed embodiments, but, on the contrary, are intended to cover modifications and equivalent arrangements that are within the spirit and scope of the described embodiments. For example, it is to be understood that the present invention contemplates that, to the extent possible, one or more features of any embodiment can be combined with one or more features of any other embodiment. Likewise, embodiments may be incorporated into systems including glove boxes and clean rooms.

1. A method of treating a chamber, comprising:
humidifying the chamber to a non-condensing relative humidity;
injecting into the chamber a sterilant gas having a boiling point less than 50° C. to a non-condensing sterilant gas concentration;
recirculating the sterilant gas through the chamber for a selected treatment period; and
purging the sterilant gas from the chamber.

2. A method as in claim 1, wherein during the treating, a positive pressure is maintained relative to ambient pressure outside the chamber.

3. A method as in claim 1, wherein during the treating, temperature in the chamber is maintained between 10° C. and 50° C.

4. A method as in claim 1, wherein the concentration of sterilant gas is selected to be sufficient to sterilize the chamber in less than 100 minutes.

5. A method as in claim 4, wherein the concentration of sterilant gas is selected to be sufficient to sterilize the chamber in less than 20 minutes.

6. A method as in claim 1, wherein the concentration of sterilant gas is controlled by controlling a duration of the injecting.

7. A method as in claim 1, wherein the concentration of sterilant gas is controlled by controlling a duration of the injecting the sterilant into a pre-chamber in fluid communication with the chamber.

8. A method as in claim 1, wherein relative humidity is maintained between 70 and 85% relative humidity.

9. A method as in claim 1, wherein relative humidity is maintained between 55 and 70% relative humidity.

10. A method as in claim 1, wherein the purging comprises injecting gas free of sterilant.

11. A method as in claim 1, wherein the purging comprises recirculating the chamber gas through a sterilant-removing filter.

12. A method as in claim 10, wherein a rate of injecting of sterilant-free gas is selected such that the concentration of sterilant gas is reduced to less than 1 ppm in 30 minutes.

13. A method as in claim 11, wherein a rate of exchanging the gas in the chamber by recirculation through a sterilant-removing filter is selected such that the concentration of sterilant gas is reduced to less than 1 ppm in 30 minutes.

14. A method of removing the sterilant from the chamber, as in claim 10, wherein the flow rate is greater than 0.5 times the chamber volume per minute.

15. A method of removing the sterilant from the chamber by injecting or recirculating air, as in claim 10, wherein the air is controlled to be below 40% RH.

16. A method as in claim 1, wherein the sterilant gas is NO₂.

17. A method as in claim 1, wherein the sterliant gas concentration is controlled by measuring pressure of the gas in a pre-chamber having a known volume and being in fluid communication with the chamber.
